(19) United States
(12) Patent Application Publication
(10) Pub. No.: US 2006/0134696 A1
(43) Pub. Date: Jun. 22, 2006

(54) Small molecule substrate based enzyme activity assays

(75) Inventors: Nghia H. Chiem, San Francisco, CA (US); John R. Gilbert, Brookline, MA (US)

Correspondence Address: LAHIVE & COCKFIELD, 28 STATE STREET, BOSTON, MA, 02109, US

(73) Assignee: Cytonome, Inc., Boston, MA (US)

(21) Appl. No.: 11/181,666

(22) Filed: Jul. 13, 2005

Related U.S. Application Data
(63) Continuation of application No. 10/027,922, filed on Dec. 21, 2001, now abandoned.
(60) Provisional application No. 60/323,962, filed on Sep. 20, 2001.

(51) Int. Cl. C40B 40/02; C12Q 1/48; C12Q 1/34; C12Q 1/42; C12Q 1/37

(52) U.S. Cl. 435/7.1; 435/15; 435/18; 435/21; 435/23

(57) ABSTRACT

The invention relates to a method of high throughput chemical analysis comprising the steps of combining one test compound with a solution comprising m enzyme(s) and n substrate(s), wherein m is an integer equal to one or greater, n is an integer equal to one or greater, and m+n≧3, incubating for a period of time said test compound within said solution, separating the chemical species in said combined solution by a chromatography step after said incubating step, and measuring the relative amounts of substrates and separately identifiable products produced therefrom by a chemical reaction catalyzed by said enzymes. The present SMSBEA assays are particularly well suited to enzyme-substrate systems in which both the substrate(s) and product(s) have mobilities such that they can be separated on short chromatography columns. The method of the invention is also particularly well suited to HTS applications in which an enzyme agonist or antagonist is sought. An advantage of the method is that the effects of a test compound on several enzymes may be analyzed simultaneously and without substantial purification of the enzyme solution, e.g., whole cell lysates.

### RELATED APPLICATIONS

This application is a continuation of U.S. patent application Ser. No. 10/027,922 filed Dec. 21, 2001, which claims priority to U.S. Provisional Patent Application No. 60/323,962 filed Sep. 20, 2001 and is related to Attorney Docket No. CVZ-001a, entitled “Microfluidic System Including a Virtual Wall Fluid Interface Port for Interfacing Fluids with the Microfluidic System”, filed herewith; Attorney Docket No. CVZ-001b, entitled “Microfluidic System Including a Virtual Wall Fluid Interface Port for Interfacing Fluids with the Microfluidic System”, filed herewith; Attorney Docket No. CVZ-001c, entitled “Microfluidic System Including a Virtual Wall Fluid Interface Port for Interfacing Fluids with the Microfluidic System”, filed herewith; and Attorney Docket No. CVZ-005, entitled “Droplet Dispensing System”, filed herewith. The contents of the foregoing patent applications are herein incorporated by reference. The contents of all references, issued patents, or published patent applications cited herein are expressly incorporated by reference.

### BACKGROUND

Enzymes are frequently critical components of biological pathways, and accordingly substantial interest exists in discovering compounds which modulate the activity of such enzymes. Compounds which act as antagonists or agonists of a particular enzyme are of interest as potential pharmacological agents. Traditional experimental searches for such agonists or antagonists were done one compound at a time, and one enzyme at a time. Modern combinatorial chemistry has accelerated the speed with new compounds, potential pharmacological agents, may be synthesized. High throughput screening (“HTS”) assays have also accelerated the speed with compounds are assayed for modulation (particularly inhibition) of enzyme activity.

Such HTS assays typically probe the effects of a test compound on a single enzyme-substrate pair, however. Complications frequently arise when such a test compound which shows promise in an in vitro HTS assay is investigated in an in vivo system. Because typical HTS assays confine themselves to a particular enzyme-substrate pair, those in vitro assays do not provide information about how such a test compound would affect other enzymes. For example, a HTS assay may identify a potent kinase inhibitor, but when such a compound is tested in vivo it is discovered that such a compound detrimentally inhibits all kinases without any specificity for the kinase of pharmacological interest.

### SUMMARY OF THE INVENTION

The present invention solves this problem by providing methods by which a test compound may be assayed simultaneously against many enzyme-substrate pairs. Such experimental data may identify compounds which are not only are potent inhibitors or stimulators of an enzyme, but may also provide data about the relative specificity of such a test compound for, e.g., inhibiting one enzyme significantly more than other similar enzymes.

The present invention relates to SMSBEA (Small Molecule Substrate Based Enzyme Activity Assays) assays in which the effect of a test compound on the activity of an enzyme in converting a substrate into a product is studied. The assay may be carried out with multiple enzymes and one substrate, and in this case the selectivity of a test compound for modulating one enzyme in preference to others may be studied. On the other hand, an assay may be carried out with one enzyme and multiple substrates, in which case the selectivity of a test compound for modulating the enzyme's ability to selectively catalyze a reaction on some of the substrates in preference to others may be studied. And finally, the assay may be carried out with several enzymes and several substrates to which a test compound is added.

In particular, the invention relates to a method of high throughput chemical analysis comprising the steps of combining one test compound with a solution comprising m enzyme(s) and n substrate(s), wherein m is an integer equal to one or greater, n is an integer equal to one or greater, and m+n≧3 (that is, there must be at least two enzymes or two substrates), incubating for a period of time said test compound within said solution, separating the chemical species in said combined solution by a chromatography step after said incubating step, and measuring the relative amounts of substrates and separately identifiable products produced therefrom by a chemical reaction catalyzed by said enzymes.

The present SMSBEA assays are particularly well suited to enzyme-substrate systems in which both the substrate(s) and product(s) have mobilities such that they can be separated on short chromatography columns, especially capillary electrophoresis (“CE”) columns, in under about 5 minutes, and in the case of CE, a standard CE column of less than about 8 cm

The method of the invention is also particularly well suited to HTS applications in which an enzyme agonist or antagonist is sought. The method of the invention permits the study of the effect of a test compound on several enzymes or substrates simultaneously. The advantages of such a method over separate assays in multiple wells are that experimental results are not degraded by variation of test compound concentration from well to well. Similarly, the data regarding the effects of a test compound (inhibition or stimulation) represent a measure of the selectivity of the test compound. To the extent that different substrates mimic different natural substrates this is valuable information about drug selectivity. Another advantage of this method, when the chromatography step is capillary electrophoresis, is that one CE channel separation can measure all the substrate/product pair ratios in one relatively quick experiment. This represents improved efficiency for HTS applications.

### DETAILED DESCRIPTION OF THE INVENTION

One class of enzyme activity assays to which the present invention is particularly suited, herein Small Molecule Substrate Based Enzyme Activity Assays (SMSBEA), are those in which an enzyme (“E”) converts a substrate (“S”) into a product (“P”). Such an assay is generally performed by incubating an enzyme, or enzyme mixture, and a substrate, or substrate mixture, together in an appropriate buffer with a test compound for a defined time. Such an incubation may be in an appropriate vessel, and several incubation experiments may be carried out in parallel. After incubation, the ratio of the substrate(s) S to the product(s) P is measured over a period of time, and the rate of conversion of S to P is a measure of the enzyme activity. The present SMSBEA assays are particularly well suited to enzyme-substrate systems in which both the substrate(s) and product(s) have mobilities such that they can be separated on short chromatography columns, especially capillary electrophoresis (“CE”) columns, in under about 5 minutes, and in the case of CE, a standard CE column of less than about 8 cm. (In a standard CE instrument the minimum column or capillary length is determined by the CE instrument and is typically longer than 8 cm. A typical example: length 27 cm, 50 um i.d. made by Polymicro Technologies, Phoenix, Ariz. Separation buffer (mobile phase) is 50 mM borate buffer at pH 9. Flow rate is typically in the pL to nL range. However, if components are separable on a shorter column, such as one less than 8 cm, then separations may be very fast as required for a HTS application, and such a CE column may be within a microfluidic chip.)

In high throughput screening, a candidate test compound may be added to an incubation vessel and the resulting change in S/P ratio in the presence of a test compound is a measure of the test compound's effectiveness as either an agonist or antagonist. A high throughput screening assay of a library of millions of candidate compounds is contemplated by the present invention.

The present invention contemplates several embodiments of SMSBEA assays: In all cases, one test compound is present (however, no test compound may be present in a control experiment). The assay may be carried out with multiple enzymes and one substrate, and in this case the selectivity of a test compound for modulating one enzyme in preference to others may be studied. On the other hand, an assay may be carried out with one enzyme and multiple substrates, in which case the selectivity of a test compound for modulating the enzyme's ability to selectively catalyze a reaction on some of the substrates in preference to others may be studied. And finally, the assay may be carried out with several enzymes and several substrates to which a test compound is added, such as in a whole cell lysate in which all of the cell's naturally occurring enzymes and substrates are present. The ability to assay an entire cellular extract represents an advantage of the present invention over conventional laboratory techniques which typically require extensive purification of an enzyme of interest before analysis. In general, the present invention may also be applied to a variety of enzyme-containing liquid samples, including solutions of compounds, whole cells or cell lysates, proteins or peptides, and particles.

In particular, the invention relates to a method of high throughput chemical analysis comprising the steps of combining one test compound with a solution comprising m enzyme(s) and n substrate(s), wherein m is an integer equal to one or greater, n is an integer equal to one or greater, and m+n≧3 (that is, there must be at least two enzymes or two substrates), incubating for a period of time said test compound within said solution, separating the chemical species in said combined solution by a chromatography step after said incubating step, and measuring the relative amounts of substrates and separately identifiable products produced therefrom by a chemical reaction catalyzed by said enzymes.

In preferred embodiments, of the method above m=1 and n≧2, or m≧2 and n=1, or m≧2 and n≧2; and m≦100 (preferably m≦50, and more preferably m≦10) or n≦100 (preferably m≦50, and more preferably n≦10).

The method may comprise an additional step of repeating the above steps with a different test compound and comparing the data obtained above to data collected from repeating the method under substantially identical conditions with the different test compound. In such a case, the first chromatogram may be quantitatively compared with the second chromatogram, such as by quantitatively comparing peak areas which have been standardized with an internal or external standard, thereby producing information about the differential selectivities of test compounds.

Similarly, the method may comprise an additional step of repeating the above method steps with no test compound, i.e. a control experiment, and comparing the data obtained above to data collected from repeating the method under substantially identical conditions without a test compound. In such a case, the first chromatogram may be quantitatively compared with the second chromatogram thereby producing information about the differential effects of a test compound, for example, in inhibiting one enzyme selectively.

The test compound may be selected from a combinatorial library, and in such a case it may be advantageous to carry out the method of the invention in a parallel fashion. For example, compound of a library may be incubated in separate wells of a standard 96 well plate with stock enzyme and substrate solutions. Therefore, the invention may be carried out multiply, that is, in parallel. Such multiple experiments may be either nearly simultaneous or sequential. Test compounds are preferably “small molecules” as the term is generally understood in the art. Such small molecules preferably have a molecular weight of less than 2500, and more preferably less than 1500. Small molecule test compounds are preferably not naturally occurring peptides, and are preferably synthetic molecules.

Some enzymes contemplated for analysis by the present invention include oxidoreductases, transferases, hydrolases, lyases, isomerases, and ligases. More particularly, synthetases, proteases, esterases, kinases and phosphatases are preferred enzymes. The reaction catalyzed by the reaction may be a hydrolysis, oxidation-reduction, metathesis, or isomerization reaction. The test compound may increase or decrease the rate of this reaction. Buffers and enzyme cofactors may also be present in the reaction medium. Similarly, internal chromatography standards may be included in the reaction medium, or added after the reaction has commenced.

The substrates are preferably compounds, either naturally occurring or synthetic, on which an enzyme catalyzes a reaction. For example, such a reaction may be an addition or removal of a phosphate group from a tyrosine residue of a peptide substrate by a kinase or phosphatase enzyme. For ease of detection of either the substrate or subsequent products, substrates may be labeled, for example with a chromophore or a radioisotope. When multiple substrates are present, each substrate may be labeled differently. Some examples of preferred optically detectable labeling reagents include fluorescamine, O-phthalaldehyde (OPA), and napthalene-2,3,-dicarboxaldehyde (NDA), fluorescein. Other labeling reagents which may be used in conjunction with the invention include dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2-(4-methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino4-methyl-2-oxo-3-chromenyl)maleimide; N-(p-(2-benzimidazolyl)-phenyl)maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone. Many such fluorescent labeling reagents are commercially available from SIGMA chemical company (Saint Louis, Mo.), Molecular Probes (Eugene, Oreg.), R&D systems (Minneapolis, Minn.), Pharmacia LKB Biotechnology. (Piscataway, N.J.), CLONTECH Laboratories, Inc. (Palo Alto, Calif.), ChemGenes Corp. (Ashland, Mass.), Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.) as well as other commercial sources known to one of skill. Such labeling may be conducted on-chip. When the chromatography step of the invention is carried out on-chip, an on-chip labeling method may also be executed on chip. See, e.g., Harrison et al. Sensors and Actuators B, 33, 105-09 (1996) for an illustrative example. Such a labeling step may be before chromatography (in which case labeled compounds are separated), or after chromatography (in which case unlabeled compounds are separated and labeled before detection).

The incubation step may be carried out for a fixed period of time. The time may be varied and an experiment run under several different time lengths. In such a case, information about reaction kinetics, and how a test compound affects the kinetics, may be learned. Likewise, the incubation step may be carried out in several parallel experiments at different temperatures giving information about reaction kinetics. Similarly, parallel incubations may be at different concentrations.

After incubation and prior to chromatography, the reaction may be stopped by heat shocking the reaction (e.g., freezing) or adding denaturing reagents such as detergents. One may desire to stop a reaction to ensure that the reaction has progressed for a controlled and definite period of time. Such methods may be particularly advantageous when multiple experiments are run simultaneously and it is undesirable, or impossible, to analyze each reaction mixture simultaneously. Such a step may be referred to as “quenching” a reaction. An example of such a quenching reagent is trichloroacetic acid.

After incubation, the substrates, if any remain, and the products are separated by chromatography and the components of the solution exiting the chromatography column are measured, preferably quantitatively. The chromatography conditions should be such that the products are separately identifiable from each other, e.g. they each are represented by separate peaks on a chromatogram for which representative peak areas may be calculated. Preferably, the chromatography step is carried out within a microfluidic chip. Chromatography systems may include electrophoresis or ion chromatography; high, medium, or low pressure liquid chromatography; or any combination thereof. Other preferred chromatography systems include high performance liquid chromatography or conventional capillary electrophoresis or capillary electrochromatography. An especially preferred chromatography system is a CE microfluidic device, such as a chip. Capillary electrophoresis chromatography columns are particularly preferred chromatography means according to the invention. Such microfluidic CE columns are described in U.S. Pat. Nos. 6,159,353, 5,976,336, and 6,258,263, each of which are incorporated herein by reference.

Recent developments in microscale chemical analysis systems made it possible to perform multi step, multi species chemical operations in such chip-based micro chemical analysis systems. See Waters et al., Anal. Chem. 70:158-162 Jan. (1998), Haswell, Analyst 122:1R-10R Jan. (1997), Jacobson et al., Anal. Chem. 66:3472-3476 Jul. (1994), Ramsey et al., Nature Med. 1:1093-1096 Oct. (1995), Manz et al., Sensors and Actuators B1:244-248 (1990) and Manz et al., J. Chromatography 593:253-258 (1992). Generally, these chip-based systems comprise ‘microfluidic’ elements, particularly capable of handling and analyzing chemical and biological specimens. The term microfluidic refers to systems or devices having a network of processing nodes, chambers and reservoirs connected by channels, in which the channels have typical cross-sectional dimensions in the range from about 0.1 μm to about 500 μm. In the art, channels having these cross-sectional dimensions are referred to as ‘microchannels’.

When CE separation is employed, the products preferably have a different mass to charge ratio than the substrates. Likewise, preferred enzyme-substrate systems for separation and analysis by CE are those which produce products which may be resolved in a capillary electrophoresis column of less than about 20 cm, preferably less than about 12 cm, and most preferably less than about 8 cm in length in under 20 minutes, preferably less than about 10 minutes, and most preferably in less than about 5 minutes. A separation which may be carried out in a conventional CE column of such dimensions may also be executed on-chip in conveniently short analysis times and with sufficient resolution.

Although it is preferred that the incubation step occur outside of a microfluidic device, the method of the invention may be carried out wholly or partially within such a microfluidic device. As mentioned above, a microfluidic CE chip may be used for the chromatography step. Similarly, the incubation step may be carried out within the microfluidic chip. In such a case, the microfluidic chip contains a reaction means, such as an incubation region, for example a channel or reservoir. A microfluidic chip should have an introduction means by which a liquid sample is introduced into the chip. Such an introduction means may be a capillary, an aperture, or a hole. An especially preferred introduction means is a ‘virtual wall’, as described in U.S. Provisional Patent Application 60/299,515, filed Jun. 20, 2001, and U.S. Utility patent applications Attorney Docket No. CVZ-001a, entitled “Microfluidic System Including a Virtual Wall Fluid Interface Port for Interfacing Fluids with the Microfluidic System”, filed herewith; Attorney Docket No. CVZ-001b, entitled “Microfluidic System Including a Virtual Wall Fluid Interface Port for Interfacing Fluids with the Microfluidic System”, filed herewith; Attorney Docket No. CVZ-001c, entitled “Microfluidic System Including a Virtual Wall Fluid Interface Port for Interfacing Fluids with the Microfluidic System”, filed herewith, the contents and teachings of which are incorporated herein. Microfluidic chips of various designs, including those with multiple incubation regions and/or multiple CE separation regions as known in the art may be used in accordance with the invention.

By performing the chemical operations in a microfluidic system, potentially a number of the mentioned desirable improvements can be realized. By down scaling dimensions, diffusional processes like heating, cooling and passive transport of species (diffusional mass-transport) proceed faster. One example is the thermal processing of liquids, which is mostly a required step in chemical synthesis and analysis. Compared to the heating and cooling of liquids in beakers as performed in a conventional laboratory setting, the thermal processing of liquids proceeds extremely fast in a microchannel due to the reduced diffusional distances. Another example is the mixing of dissolved species in a liquid, a process which is also diffusion limited. Downscaling the typical dimensions of the mixing chamber thereby reducing the typical distance to be overcome by diffusional mass-transport, will result in a drastic reduction of mixing times. Like thermal processing, the mixing of dissolved chemical species, such as reagents with a sample or precursors for a synthesis step, is an operation that is required in virtually all chemical synthesis and analysis processes.

Furthermore, by the reduction of typical dimensions separation operations are more efficient. Such an example is capillary electrophoresis, which is a separation technology based on the migration of dissolved charged species through a liquid filled capillary by the application of a longitudinal electric field. It is generally known that by reducing the cross-sectional size of the capillaries, the separation efficiency can greatly be improved, resulting in rapid separations. See Effenhauser et al., Anal. Chem. 65:2637-2642 Oct. (1993), Effenhauser et al., Anal. Chem. 66:2949-2953 Sep. (1994), Jacobson et al., Anal. Chem. 66:4127-4132 Dec. (1994) and Jacobson et al., Anal. Chem. 66:1114-1118 Apr. (1994). Accordingly, the method of the invention preferentially employs a chip-based CE separation.

Another aspect of the reduction of dimensions is the reduction of required volumes of sample, reagents, precursors and other often very expensive chemical substances. While in milliliters sized systems typically milliliter volumes of these substances are required, in microliter sized microfluidic systems only microliters are required. As a consequence also the amount of chemical waste produced during the chemical operations is reduced. Both effects of volumetric downscaling can significantly reduce costs and allow the economic operation of chemical synthesis and analysis systems.

Also due to the reduced dimensions associated with microfluidic systems, important chemical operations can be accelerated whilst at the same instance lead to a reduction of consumption of chemicals and chemical waste.

An apparatus for performing electrophoretic experiments in a highly parallel fashion is disclosed in U.S. Pat. No. 6,103,199. Here, a plurality of separation capillaries with associated wells for receiving chemical substances in fluid form, are disposed in the form of a two dimensional array. The chemical substances are dispensed from a micro titer plate into these wells by an interfacing methodology employing pressurized chambers associated with the wells to be filled. Other interfacing technologies as are known in the art may be employed.

After chromatography, the reaction components are measured, preferably quantitatively. Substrates or products of the reaction may be identified by the retention time or order of elution in conjunction with control experiments using external or internal standards. The measurement step preferably produces a quantitative chromatogram in which peaks of interest are known through appropriate control experiments are a known in the chromatography art. In accordance with one aspect of the invention, a chromatogram resulting from one experiment with a test compound is compared to a standard chromatogram or a chromatogram with another test compound. Such a comparison may be quantitative, and may be carried out using an automated computer program.

The measuring step may be spectrometry or spectroscopy. The physical parameter which is measured may be molecular mass (by mass spectrometry), chromatographic retention time, spectroscopic absorbance or emission (including fluorescence), refractive index, electrical conductivity, or radioactivity. The measurement step is preferably quantitative.

The method of the invention may be carried out on a variety of hardware platforms. In one exemplary embodiment, the method is carried out in parallel in which different test compounds are individually incubated with a standardized enzyme and substrate solutions in a 96 well plate. After a defined period of time, the reactions are quenched, a fixed amount of an internal chromatography standard is added to each reaction, and each reaction is separated by HPLC. Such a quenching step may be carried out on-chip with any reagent which substantially inhibits or interferes with the enzyme-substrate action, such as denaturing agents including detergents. Preferably such a quenching agent does not interfere with subsequent detection. Should a reaction be slow compared with the analysis time, a quenching step may not be necessary. Likewise, if a reaction is continuously monitored, quenching may not be necessary. Sample manipulation may be done by robots, autosamplers, and other routinely used laboratory automation equipment.

In another exemplary embodiment, experiments are conducted either singly or multiply, and separations are performed on a CE microfluidic chip. The incubation may be either within the chip, or in separate containers, such as a 96 well plate. When the incubation is performed outside of a chip, a quenching step may be employed, and samples may be transferred, for example by spotting a drop of the reaction solution or micropipetting a sample, into a CE chip.

In a further alternative embodiment, multiple aliquots of a reaction may be analyzed at different times during the course of a reaction.

### EXAMPLES

One Enzyme and Multiple Substrates (m=1 and n≧2)

Most enzyme are highly specific both in the nature of the natural substrates which they utilize and also in the reaction they catalyze. However, many enzymes may be used with a variety of artificial substrates when assaying for their activity in HTS application. For these enzymes and enzymes of less natural specificity this method would be advantageous. Examples of such enzymes include kinases, proteases, phosphatases and esterases which are typically assayed with small artificial peptides.

Among the advantages of such a multiple substrates approach over separate assays in multiple wells is that all reactions are carried out in the same well with the same concentration of enzyme. Therefore, well-to-well variation is reduced or eliminated, and measurement is not clouded by variation in the enzyme concentration or incubation as it would be if done in separate wells. Also, the experimental data regarding a test compound (agonist or antagonist) is also not degraded by the variation in test compound concentration from well to well. Such data indicate the selectivity of the test compound. To the extent that different test substrate mimic different natural substrates this is valuable information about drug selectivity.

If the experimental results indicate that the test compound causes the enzyme to amplify selectivity between substrates it may be an indication that the test compound affects the substrate binding site on the enzyme, as opposed to other sites on the enzyme. Alternatively, such amplified selectivity may result from selective inhibition of an enzyme by binding to any site on an enzyme including the active site. Therefore, HTS screening for this effect may detect binding site specific drug candidates. Such candidates may be more specific to that enzyme per se than non-specific drugs. For example, when assaying a test compound against ATP-dependent enzymes, the selectivity of potential ATP binding site blockers, possibly affecting many ATP-dependent enzymes, may be studied and selective inhibitors discovered.

Another advantage of this method is that one CE channel separation can measure all the substrate/product pair ratios in one relatively quick experiment. This represents improved efficiency for HTS applications.

By way of example, the effects of test compounds on the following peptide-enzyme systems may be assayed.

| | | | Kinase |
|---|---|---|---|
| Substrate # | Substrates | Structures | Products |
| Set 1 | | | |
| 1 | SPG | Gly Ser Pro Glu Pro | pSPG |
| | | Pro Pro Glu Glu Glu | |
| 2 | GPS | Gly Glu Pro Ser Pro | GPpS |
| | | Pro Pro Glu Glu Glu | |
| 3 | SPS | Gly Ser Pro Ser Pro | pSPpS |
| | | Pro Pro Glu Glu Glu | |
| Set 2 | | | |
| 4 | TY | Glu Lys Ile Gly Glu | TpY, |
| | | Gly Thr Tyr Gly Val | |
| 5 | YY | Glu Lys Ile Gly Glu | pYpY |
| | | Gly Tyr Tyr Gly Val | |

According to the example, two sets of phosphopeptide isomers (i.e. n=3) may be used in an experiment with an enzyme (i.e. m=1). Set 1 peptides have the sequence GXPXPPPEEE where at least one X is a serine residues available for phosphorylation. Set 2 peptides have the sequence EKIGEGXXGV where at least one X tyrosine available for phosphorylation. The effect of a test compound on the ability of mitogen activated protein (MAP) kinase or protein kinase A (PKA) and tyrosine kinase GST-TK, which may be obtained from Calbiochem-Novabiochem Corp. (La Jolla, Calif.), to phosphorylate a substrate is studied.

A typical reaction may in a volume of 25 μL and carried out in a microwell of a microtiter plate. A mixture of nonphosphorylated peptides from Set 1 in the Table above (50 μg/mL) and the test compound (e.g. a putative inhibitor) are incubated with either a MAP kinase or PKA kinase at room temperature. The reaction mixture for the incubation with MAP kinase may consist of 40 μg/mL MAP kinase, 20 mM HEPES buffer (pH 7.55), 5 mM MgCl₂, 5 mM β-mercaptoethanol, and 1 mM ATP. The reaction mixture for the incubation with PKA may be carried out analogously, but with a different amount of kinase (e.g. 1000 units/mL). See generally Gamble, et al., Anal. Chem. 71, 3469-76 (1999).

After a desired time of incubation, the reaction is terminated by addition of SDS or EDTA to quench the reaction. A labeling dye, e.g. fluorescamine, from a stock solution (3 mg/mL) is added and the separation of products is achieved by capillary electrophoresis on-chip. A typical separation chip consists of a set of intersecting micro-channels which have typical width of about 50 μm and an isotropically etched depth of about 15 μm. A typical separation is achieved by electrokinetically injecting a sample plug of about 150 μm into the separation channel and separation is achieved by applying voltage with an electric field of about 800 V/cm. The typical separation length is 5 cm or less. In detection, laser induced fluorescence is used with excitation and emission wavelengths selected at 390 nm and 450 nm, respectively. The separated product peaks are identified and measured from the electropherograms. The effect of the test compound on the enzyme is measured by monitoring the amount of products formed and comparing with those obtained from a controlled solution where no test compounds were used.

Multiple Enzymes and Multiple Substrates (m≧2 and n≧2)

In general, enzymatic reactions of multiple enzyme-substrate pairs in the same solution before separation may be represented by
Eₐ: Sₐ [symbol] Pₐ (i.e., Enzyme a catalyzes Substrate a into Product a)
E_{b}: S_{b} [symbol] P_{b}
E_{c}: S_{c} [symbol] P_{c}
. . .
Eₙ: Sₙ [symbol] Pₙ

The method of the present invention is particularly applicable when each enzyme reaction does not significantly affecting the others (i.e., E_{b}: Sₐ [symbol] P_{b} does not occur) and the products can be resolved by chromatographic separation.

The advantages of such a method over separate assays in multiple wells are that experimental results are not degraded by variation of test compound concentration from well to well. Similarly, the data regarding the effects of a test compound (inhibition or stimulation) represent a measure of the selectivity of the test compound. To the extent that different substrates mimic different natural substrates this is valuable information about drug selectivity.

Another advantage of this method is that one CE channel separation can measure all the substrate/product pair ratios in one relatively quick experiment. This represents improved efficiency for HTS applications.

By way of example, the experiment outlined above may be executed in like manner, but with both MAP kinase and tyrosine kinase GST-TK simultaneously (i.e. m=2) present in a mixture of peptides from both Sets 1 and 2.

Multiple Enzymes and One Substrate (m≧2 and n=1)

Compared with the example above, this method may be illustrated as Eₙ: S [symbol] Pₙ, that is several enzymes catalyze the reaction of one substrate into n products.

This method may be applied to the study of a biochemical pathway such as, for example, Arg/Cit cycle, illustrated below:

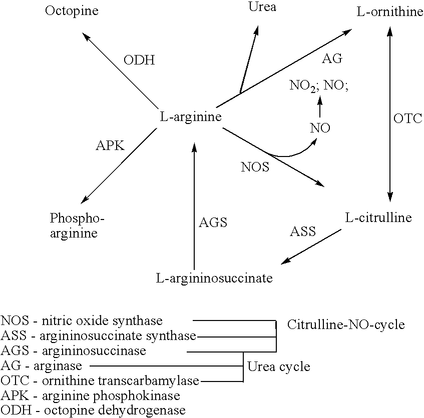

The principal substrate is L-arginine. This substrate is transformed into a variety of products by multiple enzymes, and these products may be separated and identified by CE. Therefore, according to the method of the invention one may monitor the activity of these enzymes by monitoring the concentration of their corresponding products. For example, if test compounds are added to a solution in which the above biochemical pathway has been recapitulated, the effect of that test compound on the pathway as a whole, as well as the individual products may be studied. For example, a combinatorial library may be screened for a compound which selectively inhibits arginine phosphokinase for possible use as a pharmaceutical. In such a case, the method allows to simultaneously determining is such a prospective pharmaceutical produces minimal effects on the other enzymes of the system. Such enzyme selectivity is typically desirable in pharmaceuticals. Such a method may be carried out analogously to that described above.

This method advantageously allows the screening for the presence of an enzyme in a biological sample with multiple enzymes without having to purify the enzyme first. Additionally, the effect of a test compound on multiple enzymes may be determined simultaneously.

The advantages of such an assay over separate assays in multiple wells are that all reactions are carried out in the same well with the same concentration of substrate thereby reducing or eliminating variations in substrate, enzyme, or test compound concentrations. The data regarding the effects of a test compound indicate the selectivity of the test compound as an inhibitor or stimulator. To the extent that different substrates mimic different natural substrates, this is valuable information about drug selectivity. This method is advantageously applied to cases in which a test compound produces significantly different effects on different enzymes.

If the test compound exhibits selectivity among the enzymes, it is an indication that the test compound affects the substrate binding site as opposed to other sites on the enzymes. In HTS screening applications, data indicating this effect may identify site-specific drug candidates. Such candidates may be more specific to that enzyme per se than non-specific drugs, for example, ATP binding site blockers which affect many enzymes which employ ATP as a cofactor.

As described elsewhere herein, another advantage of this method is that one CE channel separation can measure all the substrate/product pair ratios in one relatively quick experiment. This represents improved efficiency for HTS applications.

The present invention has been described relative to illustrative embodiments. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween. Those skilled in the art will recognize, or be able to ascertain using no more than routine experimentation, many equivalents to the specific embodiments of the invention described herein. Such equivalents are intended to be encompassed by the following claims.

1. A method of chemical analysis comprising the steps of
combining one test compound with a solution comprising m enzymes and n substrates complementary to said enzyme, wherein m is an integer equal to one or greater, n is an integer equal to one or greater, and m+n≧3,
incubating for a period of time said test compound within said solution,
separating the chemical species in said combined solution by a chromatography step after said incubating step, and
measuring the relative amounts of substrates and separately identifiable products produced therefrom by a chemical reaction catalyzed by said enzymes
wherein said chromatography step is carried out within a microfluidic device.

2. The method of claim 1 further comprising the step of comparing the data obtained in the measuring step to data collected from repeating the method of claim 1 under substantially identical conditions but with a different test compound.

3. The method of claim 1 further comprising the step of comparing the data obtained in the measuring step to data collected from repeating the method of claim 1 under substantially identical conditions but with no test compound.

4. The method of claim any one of claims 1-3 wherein m=1 and n≧2.

5. The method of claim any one of claims 1-3 wherein m≧2 and n=1.

6. The method of claim any one of claims 1-3 wherein m≧2 and n≧2.

7. The method of claim 1 wherein said chromatography step is electrophoresis or ion chromatography; high, medium, or low pressure liquid chromatography; or any combination thereof.

8. The method according to claim 7 wherein said chromatography step is capillary electrophoresis.

9. The method according to claim 8 wherein said enzyme catalyzes a reaction in which the products have a different mass to charge ratio than the substrates.

10. The method according to any one of claims 8 or 9 wherein said products may be resolved in a capillary electrophoresis column of less than 8 cm in length in under about 5 minutes.

11. The method according to claim 1 wherein said enzyme is an oxidoreductase, transferase, hydrolase, lyase, isomerase, or ligase.

12. The method according to claim 1 in which the combining and incubating steps of claim 1 are multiply and nearly simultaneously or sequentially executed.

13. The method according to claim 1 wherein said microfluidic device further comprises a reaction means in which said incubating step is executed.

14. The method according to claim 1 wherein m≦50.

15. The method according to claim 14 wherein m≦10.

16. The method according to claim 1 where in n≦50.

17. The method according to claim 16 wherein n≦10.

18. The method according to claim 1 wherein said test compound reduces the rate at which said enzyme converts said substrate into said product.

19. The method according to claim 1 wherein said test compound increases the rate at which said enzyme converts said substrate into said product.

20. The method according to claim 1 wherein said chemical reaction is a hydrolysis, oxidation-reduction, metathesis, or isomerization reaction.

21. The method according to claim 1 wherein said measuring step is spectrometry or spectroscopy.

22. The method according to claim 1 wherein the physical parameter which is measured is molecular mass, chromatographic retention time, spectroscopic absorbance or emission, refractive index, electrical conductivity, or radioactivity.

23. The method of claim 13 wherein said microfluidic device comprises one or more introduction means through which solutions are placed into said microfluidic device, one or more chromatography means, and one or more reaction means within which said incubation step is executed.

24. The method according to claim 23 wherein either of said introducing means is selected from a micropipette, a capillary, a virtual wall, or an aperture.

25. The method of claim 24 wherein said introduction means is a virtual wall.

26. The method of claim 23 wherein said reaction means is a reservoir or a channel.

27. The method according to claim 1 wherein said measurement step produces data which are indicative of the thermodynamics or kinetics of said chemical reaction.

28. The method according to claim 27 wherein said data are collected for reactions occurring at different temperatures or concentrations.

29. The method according to claim 27 wherein the molar concentration of said enzyme is different than the molar concentration of said substrate or test compound.

30. The method according to claim 1 wherein said enzyme is a synthetase, protease, esterase, kinase or phosphatase.

31. The method according to claim 1 wherein said substrate is the naturally occurring substrate of said enzyme, or a fragment thereof.

32. The method according to claim 1 wherein said substrate is a nonnatural or synthetic substrate for said enzyme.

33. The method according to any one of claims 31 or 32 wherein said substrate is covalently bonded to a chromophore.

34. The method according to claim 1 wherein said test compound is a member of a combinatorial library.

35. The method according to claim 1 wherein said test compound has a molecular weight of less than 2500.

36. The method according to claim 35 wherein said test compound has a molecular weight of less than 1500.

37. The method according to claim 1 wherein said test compound is not a peptide.

38. The method according to claim 1 wherein m≧2 and said measuring step produces data which indicate the relative specificity of said test compound for preferentially altering the rate of said chemical reaction with respect to one enzyme-substrate pair substantially more than for the other enzyme-substrate pair

39. The method according to claim 1 wherein m≧2 and said measuring step produces data which indicate the relative specificity of said test compound for preferentially altering the rate of reaction catalyzed by one enzyme, or binding to one enzyme, substantially in preference to other enzymes.

40. The method according to claim 1 wherein n≧2 and said measuring step produces data which indicate the relative specificity of said test compound for preferentially altering the rate of said chemical reaction with respect to one enzyme-substrate pair substantially more than for the other enzyme-substrate pair.

41. The method according to claim 1 wherein n≧2 and said measuring step produces data which indicate the relative specificity of said test compound for preferentially altering the rate of reaction of one substrate substantially in preference to other substrates.

42. The method according to claim 1 comprising an additional step of quenching said chemical reaction after incubating and prior to chromatography.

43. A method of chemical analysis comprising the steps of
combining one test compound with a solution comprising m enzymes and n substrates complementary to said enzyme, wherein m is an integer equal to one or greater, n is an integer equal to one or greater, and m+n≧3,
incubating for a period of time said test compound within said solution,
separating the chemical species in said combined solution by a capillary electrophoresis chromatography step or capillary electrochromatography step after said incubating step, and
measuring the relative amounts of substrates and separately identifiable products produced therefrom by a chemical reaction catalyzed by said enzymes
wherein said chromatography step is carried out within a microfluidic device comprising a capillary electrophoresis column.
